(19) United States
(12) Patent Application Publication
(10) Pub. No.: US 2026/0041825 A1
(43) Pub. Date: Feb. 12, 2026

(54) ASEPTIC PERITONEAL DIALYSIS TRANSFER SET

(71) Applicants: Baxter International Inc., Deerfield, IL (US); Baxter Healthcare SA, Glattpark (Opfikon) (CH)

(72) Inventor: Raghavendra Hariharapura NINGEGOWDA, Bangalore Karnataka (IN)

(21) Appl. No.: 19/100,723

(22) PCT Filed: Jul. 26, 2023
(86) PCT No.: PCT/US2023/028689

§ 371 Date: Feb. 3, 2025

(30) Foreign Application Priority Data
Aug. 2, 2022 (IN) 202241044175

(51) Int. Cl. A61M 1/14; A61M 1/28

(52) U.S. Cl. CPC A61M 1/159; A61M 1/28

(57) ABSTRACT

A peritoneal dialysis (“PD”) transfer set includes a base having at least one slot; a slider extending within the base and including an elongated tube configured to carry PD fluid, the elongated tube extending to a head of the slider, the head including at least one lug extending through the at least one slot and further including a connector for connecting to a mating patient line connector; and a shroud barrel including at least one helical groove and extending around a portion of the base so that the at least one helical groove receives the at least one lug, wherein a user may rotate the shroud barrel including the at least one helical groove such that the at least one lug extends along the at least one helical groove, the at least one slot constraining movement of the at least one lug and the slider to being a translational movement.

### BACKGROUND

The present disclosure relates generally to medical fluid treatments and in particular to peritoneal dialysis (“PD”) treatments.

Due to various causes, a person's renal system can fail. Renal failure produces several physiological derangements. It is no longer possible to balance water and minerals or to excrete daily metabolic load. Toxic end products of metabolism, such as, urea, creatinine, uric acid and others, may accumulate in a patient's blood and tissue.

Reduced kidney function and, above all, kidney failure is treated with dialysis. Dialysis removes waste, toxins and excess water from the body that normal functioning kidneys would otherwise remove. Dialysis treatment for replacement of kidney functions is critical to many people because the treatment is lifesaving.

One type of kidney failure therapy is Hemodialysis (“HD”), which in general uses diffusion to remove waste products from a patient's blood. A diffusive gradient occurs across the semi-permeable dialyzer between the blood and an electrolyte solution called dialysate or dialysis fluid to cause diffusion.

Hemofiltration (“HF”) is an alternative renal replacement therapy that relies on a convective transport of toxins from the patient's blood. HF is accomplished by adding substitution or replacement fluid to the extracorporeal circuit during treatment. The substitution fluid and the fluid accumulated by the patient in between treatments is ultrafiltered over the course of the HF treatment, providing a convective transport mechanism that is particularly beneficial in removing middle and large molecules.

Hemodiafiltration (“HDF”) is a treatment modality that combines convective and diffusive clearances. HDF uses dialysis fluid flowing through a dialyzer, similar to standard hemodialysis, to provide diffusive clearance. In addition, substitution solution is provided directly to the extracorporeal circuit, providing convective clearance.

Most HD, HF, and HDF treatments occur in centers. A trend towards home hemodialysis (“HHD”) exists today in part because HHD can be performed daily, offering therapeutic benefits over in-center hemodialysis treatments, which occur typically bi- or tri-weekly. Studies have shown that more frequent treatments remove more toxins and waste products and render less interdialytic fluid overload than a patient receiving less frequent but perhaps longer treatments. A patient receiving more frequent treatments does not experience as much of a down cycle (swings in fluids and toxins) as does an in-center patient, who has built-up two or three days' worth of toxins prior to a treatment. In certain areas, the closest dialysis center can be many miles from the patient's home, causing door-to-door treatment time to consume a large portion of the day. Treatments in centers close to the patient's home may also consume a large portion of the patient's day. HHD can take place overnight or during the day while the patient relaxes, works or is otherwise productive.

Another type of kidney failure therapy is peritoneal dialysis (“PD”), which infuses a dialysis solution, also called dialysis fluid or PD fluid, into a patient's peritoneal chamber via a catheter. The PD fluid comes into contact with the peritoneal membrane in the patient's peritoneal chamber. Waste, toxins and excess water pass from the patient's bloodstream, through the capillaries in the peritoneal membrane, and into the PD fluid due to diffusion and osmosis, i.e., an osmotic gradient occurs across the membrane. An osmotic agent in the PD fluid provides the osmotic gradient. Used PD fluid is drained from the patient, removing waste, toxins and excess water from the patient. This cycle is repeated, e.g., multiple times.

There are various types of peritoneal dialysis therapies, including continuous ambulatory peritoneal dialysis (“CAPD”), automated peritoneal dialysis (“APD”), tidal flow dialysis and continuous flow peritoneal dialysis (“CFPD”). CAPD is a manual dialysis treatment. Here, the patient manually connects an implanted catheter to a drain to allow used PD fluid to drain from the patient's peritoneal cavity. The patient then switches fluid communication so that the patient catheter communicates with a bag of fresh PD fluid to infuse the fresh PD fluid through the catheter and into the patient. The patient disconnects the catheter from the fresh PD fluid bag and allows the PD fluid to dwell within the patient's peritoneal cavity, wherein the transfer of waste, toxins and excess water takes place. After a dwell period, the patient repeats the manual dialysis procedure, for example, four times per day. Manual peritoneal dialysis requires a significant amount of time and effort from the patient, leaving ample room for improvement.

APD is similar to CAPD in that the dialysis treatment includes drain, fill and dwell cycles. APD machines, however, perform the cycles automatically, typically while the patient sleeps. APD machines free patients from having to manually perform the treatment cycles and from having to transport supplies during the day. APD machines connect fluidly to an implanted catheter, to a source or bag of fresh PD fluid and to a fluid drain. APD machines pump fresh PD fluid from a dialysis fluid source, through the catheter and into the patient's peritoneal chamber. APD machines also allow for the PD fluid to dwell within the chamber and for the transfer of waste, toxins and excess water to take place. The source may include multiple liters of dialysis fluid, including several solution bags.

APD machines pump used PD fluid from the patient's peritoneal cavity, though the catheter, to drain. As with the manual process, several drain, fill and dwell cycles occur during dialysis. A “last fill” may occur at the end of the APD treatment. The last fill fluid may remain in the peritoneal chamber of the patient until the start of the next treatment, or may be manually emptied at some point during the day.

The patient for both CAPD and APD is provided with a transfer set, which is connected to the patient's indwelling catheter located within the patient's peritoneal cavity. The transfer set connects to the patient line during treatment, which allows fresh PD fluid to be delivered to and used PD fluid to be removed from the patient. One known transfer set 110 is illustrated in prior art FIG. 1. Known transfer set 110 includes a body 112 that threads at one end to a port adapter 114 and at an opposing end to a sleeve 116. Port adapter 114 is fitted with a cap 118. Cap 118 is removed from port adapter 114 to allow a patient line connector of the patient line (not illustrated) to be connected to connection end 114c of port adapter 114 of the patient's transfer set. A transfer set line 120 extends from a hose-barbed end of port adapter 114 to the patient's indwelling catheter (not illustrated).

The patient line connector is a male luer connector, while the connection end 114c of port adapter 114 as illustrated is a female luer connector. The connection of the male luer patient line connector to female luer connection end 114c is the primary source of touch contamination. Touch contamination is one of the main causes of Peritonitis. Peritonitis is the most common infection and reason for failure of technique for PD. Peritonitis is a leading cause of PD patient dropout in the first 90 to 120 days of therapy. A need exists accordingly for an improved PD transfer set that reduces the potential for touch contamination, especially at the connection interface between the male luer patient line connector and the female luer connection end 114c of port adapter 114.

### SUMMARY

The present disclosure sets forth an improved peritoneal dialysis (“PD”) transfer set. The PD transfer set in one embodiment includes four components, namely, a base, a slider, a shroud barrel and a minicap. Any one or more or all of the components of the transfer set may be formed, e.g., molded, from a thermoplastic, such as polyetherimide (“PEI”), polyethersulfone (“PES”), polyamide/nylon (“PA”), acrylonitrile butadiene styrene (“ABS”), polycarbonate (“PC”), polyvinylchloride (“PVC”) or polyetheretherketone (“PEEK”).

The base includes a larger diameter cylindrical portion, which is oriented towards the patient during use. The larger diameter cylindrical portion is provided with serrations in one embodiment for a user such as the patient to grasp the cylindrical portion and maneuver the transfer set. The larger diameter cylindrical portion is open at its outer end oriented towards the patient and allows flexible tubing, e.g., silicone tubing, to extend from the patient's indwelling catheter into the transfer set. The larger diameter cylindrical portion at its other end includes a circular, washer-shaped flange wall that extends inward from the larger diameter portion to a central hole or opening. A port having an inner diameter that is the same or perhaps slightly bigger than the diameter of the hole extends at least partway through and within the larger diameter portion. The port as illustrated herein is sized in one embodiment to accept an elongated tube of the slider. The outer diameter of the elongated tube of the slider may be sized such that tubing, e.g., silicone tubing, extending from the patient's indwelling catheter seals over an outside of the elongated tube of the slider.

The base also includes a tubular portion that extends from, e.g., is formed with, the larger diameter cylindrical portion. The tubular portion extends from the circular, washer-shaped flange wall in an opposite direction as the port. In one embodiment, the tubular portion of the base is formed having a smaller diameter than the larger diameter cylindrical portion and a larger diameter than the port. A length of the tubular portion of the base is in one embodiment large enough to accept the sliding movement of a head of the slider. To that end, the tubular portion of the base is provided with at least one, and in one embodiment two, straight through-slots that extend all the way through a wall of the tubular portion. The straight through-slots are sized to receive lugs, e.g., a lug for each slot, wherein the lugs extend outwardly from the head of the slider. In one embodiment, the base including the tubular portion and the slider do not rotate during actuation. The slider including the lugs instead slides along the base, wherein the through-slots provide translational movement guides for the slider.

In addition to the through-slots of the tubular portion, the port of the base also acts as a guide for the movement of the slider. The port accepts the elongated tube of the slider and during actuation restrains the elongated tube radially as the elongated tube slides within the port.

A cycler end of the tubular portion of the base of the transfer set is in one embodiment provided with female threads that threadingly engage with male threads of the minicap. The minicap seals the interior of the transfer set during sterilization, transport and storage prior to use. The minicap is removed and discarded in one embodiment to connect the transfer set for treatment.

The slider of the transfer set as mentioned herein includes, in one embodiment, an elongated tube that extends to a head of the slider. A diameter of the elongated tube is sized to fit relatively snugly within the port of the base of the transfer set and to fit sealingly within the flexible tubing extending from the patient's indwelling catheter. Although not illustrated, an end of the elongated tube may be formed with a connecter, e.g., luer type connector, which mates with a connector, e.g., luer type connector, provided at the end of the flexible tubing extending from the patient's indwelling catheter. The larger diameter cylindrical portion of the base is large enough to accept the mating connectors of the elongated tube and the indwelling catheter tubing if provided.

The head of the slider includes an inner portion having a female luer connector including male threads and a tapered female inner wall. The female luer connector is sized in one embodiment to mate with a standard male luer, which is a standard patient line connector located at the end of a patient line extending, e.g., from a PD machine or cycler. The male luer connector of the patient line connector includes female threads that mate with the male threads of the female luer connector of the head of the slider. The male luer connector also includes a tapered port that sealingly engages the tapered female inner wall of the female luer connector of the head of the slider.

The head of the slider includes a transverse wall that extends outwardly from the inner portion of the head. The transverse extends to an outer cylindrical wall of the head of the slider. The transverse wall, cylindrical wall and inner portion of the head of slider are in one embodiment formed, e.g., molded, as one piece with the elongated tube of the slider. The radial length of the transverse wall and an outer diameter of the cylindrical wall are sized so that the cylindrical wall fits relatively snugly within in inner surface of the tubular portion of the base.

The one or more lug, e.g., two lugs at 180° from each other, extend from an outer surface of the cylindrical wall of the head of the slider. The diameter of the at least one lug is sized to fit snugly within the straight through-slots of the tubular portion of the base. The height of the at least one lug is sized so as to extend all the way through the at least one straight through-slot of the tubular portion of the base, enough so as to be able to extend into at least one helical groove of the shroud barrel. As illustrated, the location of the transverse wall extending to a middle portion of the cylindrical wall of the head of the slider enables the cylindrical wall to flex. The flexing of the cylindrical wall enables the at least one lug to be snap-fitted within the mating at least one through-slot of the tubular portion of the base during manufacturing. After snap-fitting the at least one lug into the mating at least one through-slot, the slider is slidingly constrained within the base of the transfer set.

The shroud barrel as illustrated is located generally around an outside of the tubular portion of the base. The shroud barrel is formed to define at least one blind helical groove that in an embodiment extends at least twenty, e.g., twenty-four, millimeters (about one inch) around an inner surface of the shroud barrel. The end of the at least one lug of the slider extends into the at least one blind helical groove of the shroud barrel. In the illustrated embodiment, the helical grooves are blind and do not extend all the way through the wall of the shroud barrel, which allows the shroud barrel to better provide a sterile barrier against touch contamination. It should be appreciated however that the at least one helical groove does not have to be blind and that the movement of the slider during actuation would operate as described if the at least one helical groove instead extended all the way through the wall of the shroud barrel.

The at least one blind helical groove is oriented in one embodiment such that when the user or patient rotates the shroud barrel in a clockwise direction, the slider is advanced translationally such that the female luer connector of the head of the slider is extended towards a connection location with the male luer connector of the patient line connector. Likewise, when the user or patient rotates the shroud barrel in a counterclockwise direction, the slider is reversed translationally such that the female luer connector of the head of the slider is retracted from the connection location. In an alternative embodiment, the orientation is reversed such that counterclockwise rotation causes slider translation towards connection and clockwise rotation causes slider translation away from connection.

In an embodiment, the base of the transfer set of the present disclosure does not move during activation (or reverse activation). The serrations of the larger diameter cylindrical portion of the base provide a convenient location for the user or patient to grasp and hold the transfer set steady. The slider translates during activation (or reverse activation) relative to the base. The shroud barrel is rotated by the user or patient's other hand relative to the base. If the tubular portion of the base were not provided, the rotation of the shroud barrel would simply rotate the at least one engaged lug and the remainder of the slider and no translation would occur. The at least one straight through-slot of the tubular portion of the base however prevents the slider from rotating and instead forces the slider to translate as the at least one engaged lug moves through the rotating at least one corresponding helical groove.

In light of the disclosure set forth herein, and without limiting the disclosure in any way, in a first aspect of the present disclosure, which may be combined with any other aspect, or portion thereof, a peritoneal dialysis (“PD”) transfer set includes a base including at least one slot; a slider extending within the base, the slider including an elongated tube configured to carry PD fluid, the elongated tube extending to a head of the slider, the head including at least one lug extending through the at least one slot, the head further including a connector for connecting to a mating patient line connector; and a shroud barrel including at least one helical groove, the shroud barrel extending around a portion of the base so that the at least one helical groove receives the at least one lug, wherein a user may rotate the shroud barrel including the at least one helical groove such that the at least one lug extends along the at least one helical groove, the at least one slot constraining movement of the at least one lug and the slider to being a translational movement.

In a second aspect of the present disclosure, which may be combined with any other aspect, or portion thereof, the base includes a larger diameter cylindrical portion configured for the user to hold while the user rotates the shroud barrel, the base further including a tubular portion defining the at least one slot.

In a third aspect of the present disclosure, which may be combined with any other aspect, or portion thereof, the base includes a port located within the larger diameter cylindrical portion, the port in fluid communication with the elongated tube, the port positioned and arranged to communicate fluidly with patient catheter tubing.

In a fourth aspect of the present disclosure, which may be combined with any other aspect, or portion thereof, an end of the tubular portion is provided with threads for mating with threads of a cap configured to seal the transfer set prior to treatment.

In a fifth aspect of the present disclosure, which may be combined with any other aspect, or portion thereof, an inner diameter of the tubular portion is sized to fit closely around the head of the slider.

In a sixth aspect of the present disclosure, which may be combined with any other aspect, or portion thereof, the tubular portion of the base and the shroud barrel are positioned and arranged to deter touch contamination of a connection between the slider connector and the patient line connector.

In a seventh aspect of the present disclosure, which may be combined with any other aspect, or portion thereof, the at least one slot is a straight slot.

In an eighth aspect of the present disclosure, which may be combined with any other aspect, or portion thereof, the connector of the slider is a luer connector.

In a ninth aspect of the present disclosure, which may be combined with any other aspect, or portion thereof, the slider includes an inner portion providing the connector, a transverse wall extending from the inner portion to a cylindrical wall, the at least one lug extending from the cylindrical wall.

In a tenth aspect of the present disclosure, which may be combined with any other aspect, or portion thereof, the shroud barrel extends around the tubular portion of the base.

In an eleventh aspect of the present disclosure, which may be combined with any other aspect, or portion thereof, the at least one helical groove is a blind at least one helical groove.

In a twelfth aspect of the present disclosure, which may be combined with any other aspect, or portion thereof, the at least one helical groove is formed on an inner surface of shroud barrel.

In a thirteenth aspect of the present disclosure, which may be combined with any other aspect, or portion thereof, the at least one helical groove extends at least at least twenty millimeters along the shroud barrel.

In a fourteenth aspect of the present disclosure, which may be combined with any other aspect, or portion thereof, the PD transfer set is configured such that an end of the at least one helical groove is positioned and arranged so as to allow the connector of the slider to meet the patient line connector, and wherein further rotating of the shroud barrel threads the patient line connector into the connector of the slider.

In a fifteenth aspect of the present disclosure, which may be combined with any other aspect, or portion thereof, the PD transfer set is configured such that an end of the at least one helical groove is positioned and arranged so as to allow the connector of the slider to meet the patient line connector, and wherein rotating the patient line connector threads the patient line connector into the connector of the slider.

In a sixteenth aspect of the present disclosure, which may be combined with any other aspect, or portion thereof, the PD transfer set is configured such that rotating the shroud barrel in a first direction translates the connector of the slider towards the patient line connector and rotating the shroud barrel in a second direction translates the connector of the slider away from the patient line connector.

In a seventeenth aspect of the present disclosure, which may be combined with any other aspect, or portion thereof, a peritoneal dialysis (“PD”) transfer set comprises a base including at least one slot; a slider extending within the base, the slider configured to carry PD fluid, the slider including at least one lug extending through the at least one slot and a connector for connecting to a mating patient line connector; and a shroud barrel including at least one helical groove, the shroud barrel extending around a portion of the base so that the at least one helical groove receives the at least one lug, wherein a user may rotate the shroud barrel including the at least one helical groove such that the at least one lug extends along the at least one helical groove, the at least one slot constraining movement of the at least one lug and the slider to being a translating movement, and wherein rotating the shroud barrel in a first direction translates the connector of the slider towards the patient line connector and rotating the shroud barrel in a second direction translates the connector of the slider away from the patient line connector.

In an eighteenth aspect of the present disclosure, which may be combined with any other aspect, or portion thereof, the at least one helical groove includes at least one rounded protrusion positioned and arranged to provide a temporary stop that releasably maintains the at least one lug and slider in a location towards the patient line connector or in a location away from the patient line connector.

In a nineteenth aspect of the present disclosure, which may be combined with any other aspect, or portion thereof, a peritoneal dialysis (“PD”) connection method includes providing a shroud barrel including at least one helical groove; providing a tubular portion including at least one slot; providing a connector through which PD fluid flows, the connector including at least one lug extending through the at least one slot and into the least one helical groove; and enabling the shroud barrel to be rotated to rotate the at least one helical groove so as to cause movement of the at least one lug and the connector, and wherein the at least one slot constrains the movement of the at least one lug and the connector to being a translational movement of the connector towards a mating connector for connection or away from the mating connector after disconnection.

In a twentieth aspect of the present disclosure, which may be combined with any other aspect, or portion thereof, the PD connection method includes enabling the shroud barrel to be rotated in a first direction to translate the connector towards the mating connector for connection and in a second direction to translate the connector away from the mating connector after disconnection.

In a twenty-first aspect of the present disclosure, which may be combined with any other aspect, or portion thereof, any of the features, functionality and alternatives described in connection with any one or more of FIGS. 2 to 11 may be combined with any of the features, functionality and alternatives described in connection with any other of FIGS. 2 to 11.

In light of the above aspects and the present disclosure set forth herein, it is an advantage of the present disclosure to provide an improved peritoneal dialysis (“PD”) transfer set.

It is another advantage of the present disclosure to provide an improved PD transfer set that reduces touch contamination.

It is a further advantage of the present disclosure to provide an improved PD transfer set that reduces the risk of Peritonitis.

It is yet another advantage of the present disclosure to provide an improved PD transfer set that helps fluid transfer to take place in a more sterile environment at the patient connection end of the transfer set, which is isolated from external contamination.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE FIGURES

FIG. 1 is a sectioned perspective view of a prior art peritoneal dialysis (“PD”) transfer set.

FIG. 2 is a sectioned elevation view of one embodiment of a PD transfer set of the present disclosure, wherein the slider is retracted within a base and shroud barrel of the transfer set.

FIG. 3 is a sectioned perspective view of one embodiment of a PD transfer set of the present disclosure, wherein the slider is extended within a base and shroud barrel of the transfer set for connection to a patient line connector.

FIG. 4 is a sectioned perspective view of one embodiment of the PD transfer set of the present disclosure showing lugs of a slider extending through the slots of the base and into the grooves of the shroud barrel.

FIG. 5 is a perspective view of one embodiment of a base of the PD transfer set of the present disclosure.

FIG. 6 is a perspective view of one embodiment of a portion of a slider of the PD transfer set of the present disclosure.

FIGS. 7 and 8 are perspective and sectioned perspective views, respectively, of one embodiment of a shroud barrel of the PD transfer set of the present disclosure.

FIG. 9 is a sectioned elevation view of one embodiment of a PD transfer set of the present disclosure, wherein a patient line connector has been connected to the transfer set.

FIG. 10 is a sectioned elevation view of one embodiment of a PD transfer set of the present disclosure, wherein PD fluid is flowing through the transfer set between a PD machine or cycler and the patient.

FIG. 11 is a sectioned elevation view of one embodiment of a PD transfer set of the present disclosure, wherein a patient line connector has been removed from the transfer set.

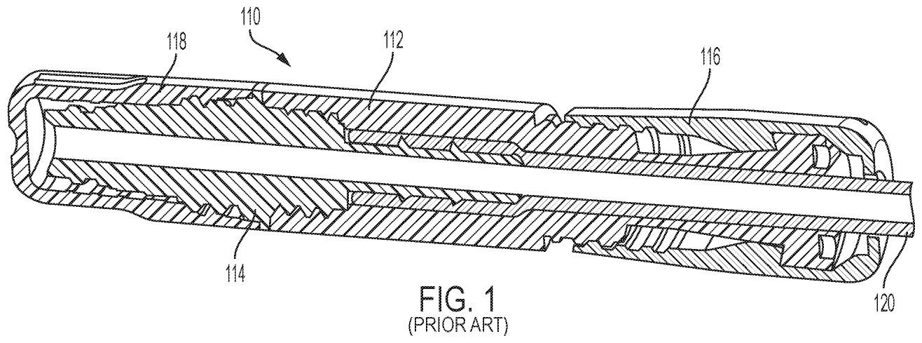

### DETAILED DESCRIPTION

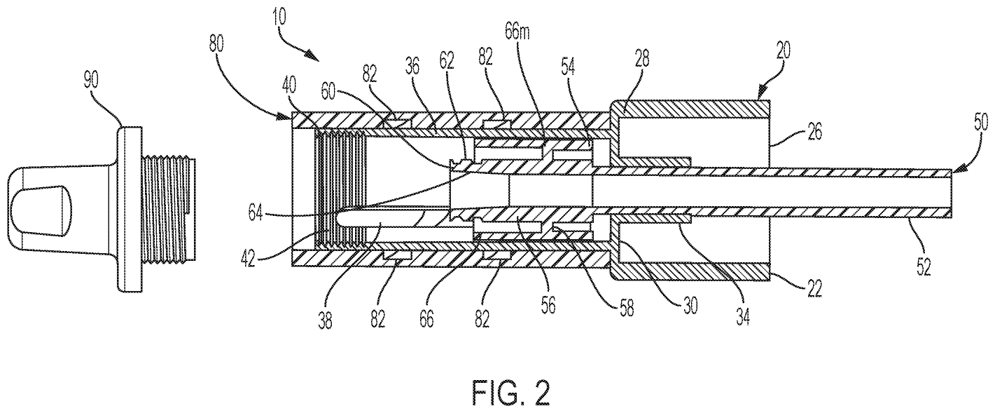

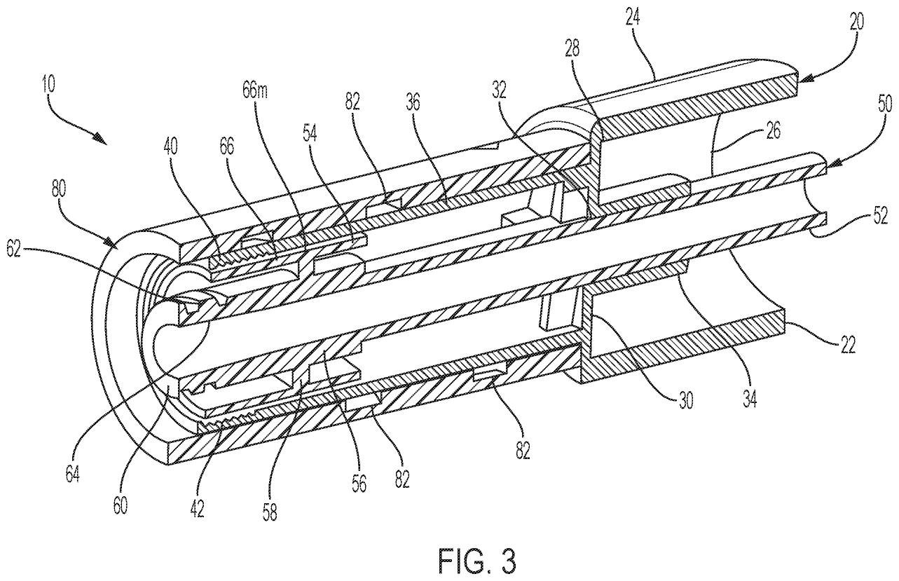

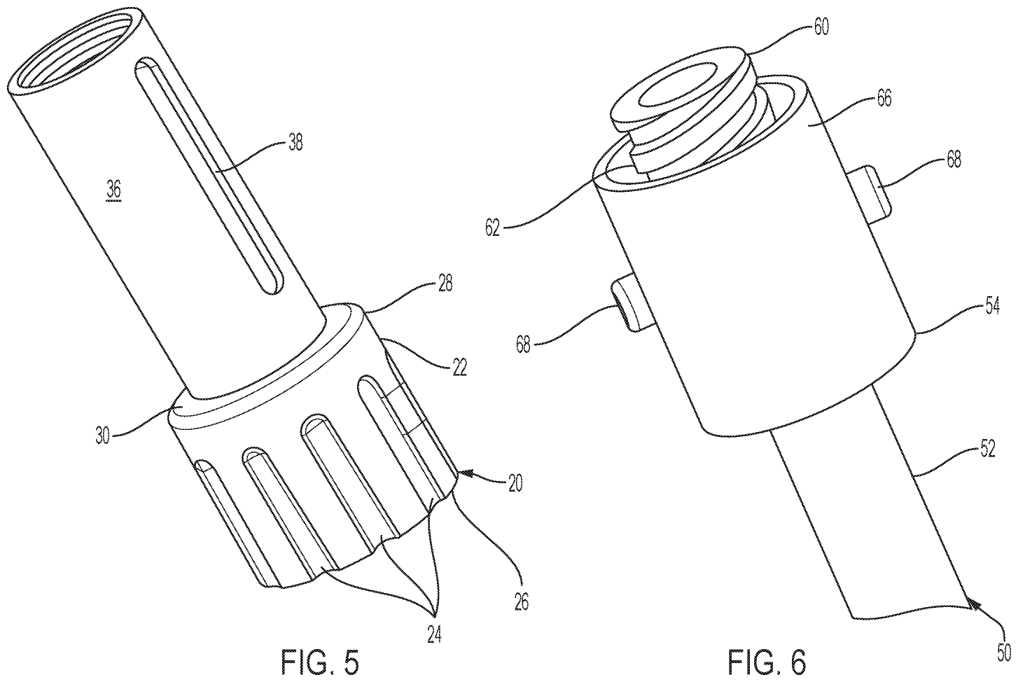

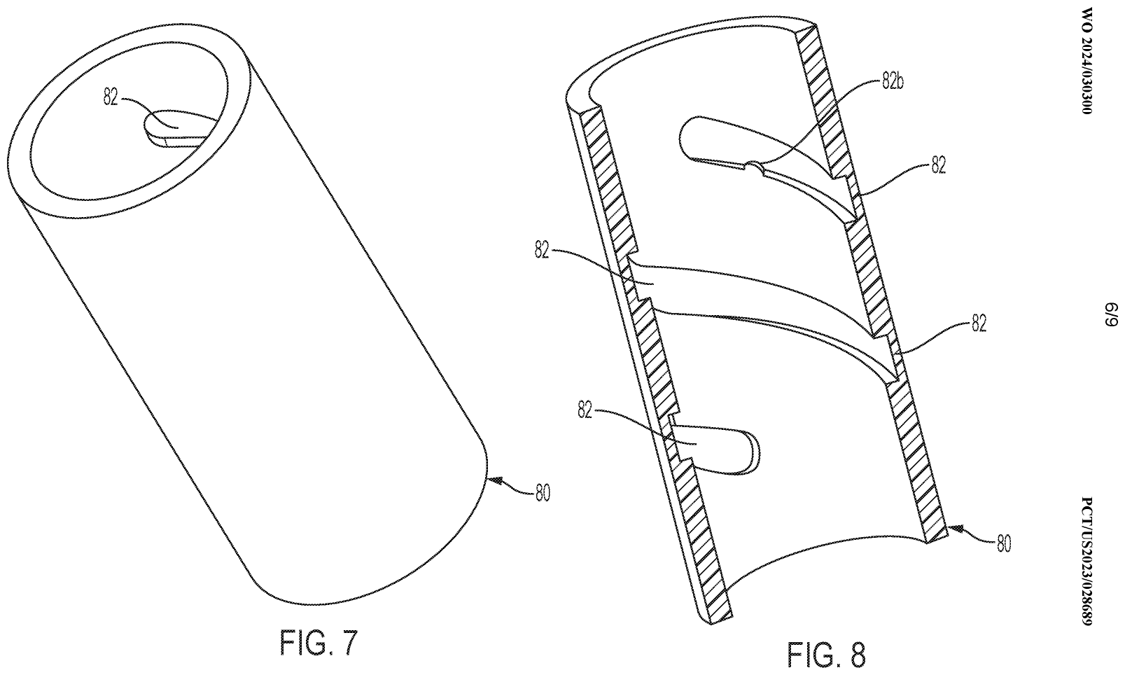

Referring again to the drawings and in particular to FIGS. 2 to 11, an improved peritoneal dialysis (“PD”) transfer set 10 is provided. PD transfer set 10 in one embodiment includes four components, namely, a base 20, a slider 50, a shroud barrel 80 and a minicap 90. Any one or more or all of the components of transfer set 10 may be formed, e.g., molded, from a thermoplastic, such as polyetherimide (“PEI”), polyethersulfone (“PES”), polyamide/nylon (“PA”), acrylonitrile butadiene styrene (“ABS”), polycarbonate (“PC”), polyvinylchloride (“PVC”) or polyetheretherketone (“PEEK”).

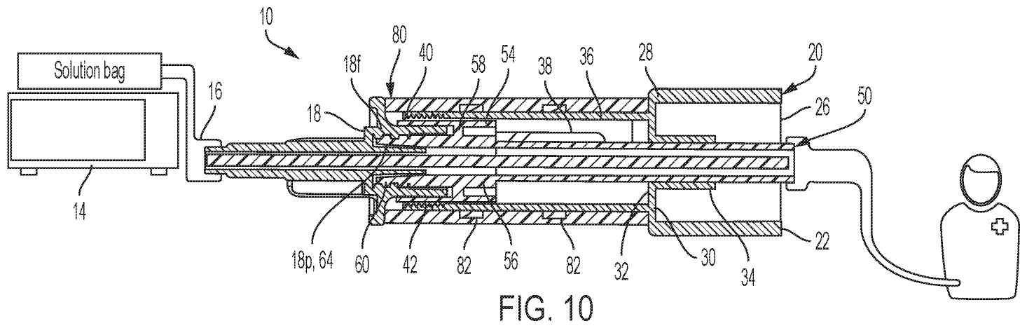

FIGS. 2, 3, 4, 5 and 9 to 11 illustrate base 20. Base 20 includes a larger diameter cylindrical portion 22, which is oriented towards the patient during use. Larger diameter cylindrical portion 22 is provided with serrations 24 in one embodiment for a user such as the patient to grasp cylindrical portion 22 and maneuver transfer set 10. Larger diameter cylindrical portion 22 is open at its outer end 26 oriented towards the patient and allows flexible tubing, e.g., silicone tubing 12 (see FIG. 10), to extend from the patient's indwelling catheter into transfer set 10. Larger diameter cylindrical portion 22 at its other end 28 includes a circular, washer-shaped flange wall 30 that extends inward from the larger diameter portion to a central hole or opening 32. A port 34 having an inner diameter that is the same or perhaps slightly bigger than the diameter of the hole extends at least partway through and within larger diameter portion 22. Port 34 as illustrated herein is sized in one embodiment to accept an elongated tube 52 of slider 50. The outer diameter of the elongated tube 52 of slider 50 may be sized such that tubing 12, e.g., silicone tubing, extending from the patient's indwelling catheter seals over an outside of the elongated tube 52 of slider 50 (see FIG. 10).

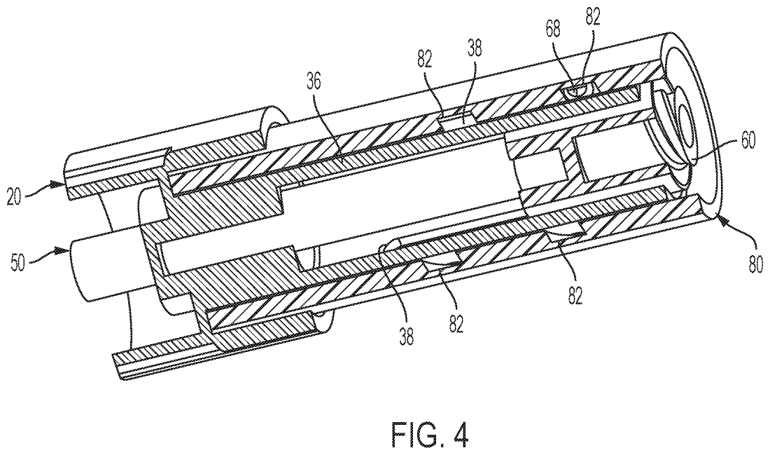

Base 20 also includes a tubular portion 36 that extends from, e.g., is formed with, larger diameter cylindrical portion 22. Tubular portion 36 extends from circular, washer-shaped flange wall 30 in an opposite direction as port 34. In one embodiment, tubular portion 36 of base 20 is formed having a smaller diameter than larger diameter cylindrical portion 22 and a larger diameter than port 34. A length of tubular portion 36 of base 20 is in one embodiment large enough to accept the sliding movement of a head of slider 50. To that end, tubular portion 36 of base 20 is provided with at least one, and in one embodiment two, straight through-slots 38 (FIGS. 2, 5, and 9 to 11) that extend all the way through a wall of the tubular portion. Straight through-slots 38 are sized to receive lugs 68 (FIGS. 4, 6), e.g., a lug for each slot, wherein lugs 68 extend outwardly from a head 54 of slider 50. In one embodiment, base 20 including tubular portion 36 and slider 50 do not rotate during actuation. Slider 50 including lugs 68 instead slides along base 20, wherein through-slots 38 provide translational movement guides for slider 50.

In addition to slots 38 of tubular portion 36, port 34 of base 20 also acts as a guide for the movement of slider 50. Port 34 accepts the elongated tube 52 of slider 50 and during actuation restrains the elongated tube radially as the elongated tube slides within the port.

A cycler end 40 of tubular portion 36 of base 20 of transfer set 10 is in one embodiment provided with female threads 42 that threadingly engage with male threads 92 of minicap 90. Minicap 90 seals the interior of transfer set 10 during sterilization, transport and storage prior to use. Minicap 90 is removed and discarded in one embodiment to connect transfer set 10 for treatment. A new minicap 90 may be replaced after treatment when the patient line is removed, wherein threads 92 of the new minicap are coated or covered with an antiseptic agent.

FIGS. 2, 3, 4, 6 and 9 to 11 illustrate slider 50. Slider 50 of transfer set 10 as mentioned herein includes, in one embodiment, an elongated tube 52 that extends to a head 54 of slider 50. A diameter of elongated tube 52 is sized to fit relatively snugly within port 34 of base 20 of transfer set 10 and to fit sealingly within flexible tubing 12 (FIG. 10) extending from the patient's indwelling catheter. Although not illustrated, an end of elongated tube 52 may be formed with a connecter, e.g., luer type connector, which mates with a connector, e.g., luer type connector, provided at the end of flexible tubing 12 extending from the patient's indwelling catheter. Larger diameter cylindrical portion 22 of base 20 as illustrated is large enough to accept the mating connectors of elongated tube 52 and of indwelling catheter tubing 12 if provided.

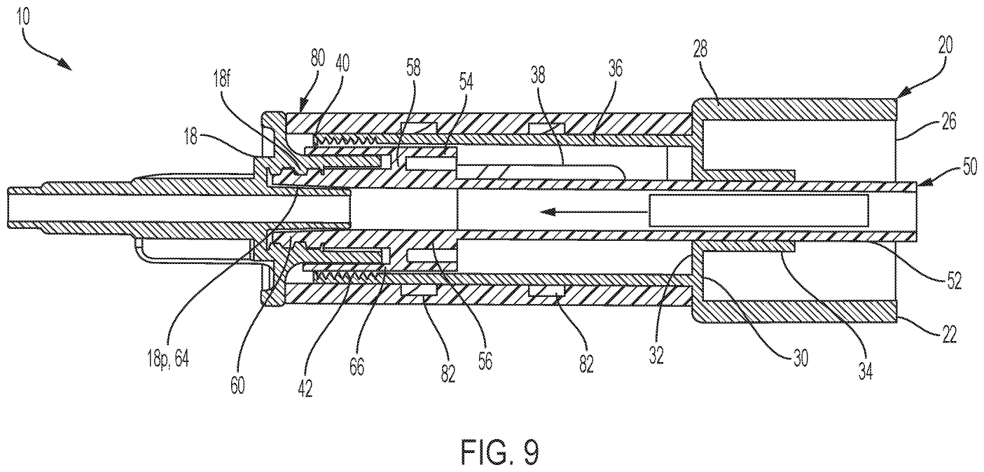

Head 54 of slider 50 includes an inner portion 56 having a female luer connector 60 including male threads 62 and a tapered female inner wall 64 (FIGS. 2, 3, 6). Female luer connector 60 is sized in one embodiment to mate with a standard male luer connector 18 (FIGS. 9 to 11), which is a standard patient line connector located at the end of a patient line 16 extending, e.g., from a PD machine or cycler 14 (FIG. 10). Male luer connector 18 includes female threads 18f that mate with male threads 62 of female luer connector 60 of the head 54 of slider 50. Male luer connector 18 also includes a tapered port 18p that sealingly engages tapered female inner wall 64 of female luer connector 60 of the head 54 of slider 50 to create a fluid-tight connection between transfer set 10 and patient line connector 18. Connection between transfer set 10 and patient line connector 18 in alternative embodiments may a connection other than a luer connection, such as a different type of threaded, and/or may involve the use of a compressible gasket.

Head 54 of slider 50 includes a transverse wall 58 that extends outwardly from the inner portion 56 of head 54. Transverse wall 58 extends to an outer cylindrical wall 66 of the head 54 of slider 50. Transverse wall 58, cylindrical wall 66 and inner portion of the head 54 of slider 50 are in one embodiment formed, e.g., molded, as one piece with elongated tube 52 of slider 50. The radial length of transverse wall 58 and an outer diameter of cylindrical wall 66 are sized in one embodiment so that cylindrical wall 66 fits relatively snugly within in inner surface of tubular portion 36 of base 20.

The one or more lug 68 (FIGS. 4 and 6), e.g., two lugs 68 positioned 180° from each other, extend from an outer surface of cylindrical wall 66 of the head 54 of slider 50. Lugs 68 may be formed with or attached, e.g., threaded, to cylindrical wall 66. The diameter of at least one lug 68 is sized to fit snugly within the straight through-slots 38 of tubular portion 36 of base 20. The height of at least one lug 68 is sized so as to extend all the way through the at least one straight through-slot 38 of tubular portion 36 of base 20, enough so as to be able to extend into at least one helical groove 82 of shroud barrel 80. As illustrated, the location of transverse wall 58 extending to a middle portion 66m of cylindrical wall 66 of the head 54 of slider 50 enables the cylindrical wall to flex. The flexing of cylindrical wall 66 enables the at least one lug 68 to be snap-fitted within the mating at least one through-slot 38 of tubular portion 36 of base 20 during manufacturing. After snap-fitting at least one lug 68 into the mating at least one through-slot 38, slider 50 is slidingly constrained within base 20 of transfer set 10. At least one lug 68 is in one embodiment spring-loaded such that it may be compressed during manufacturing and then snap through at least one slot 38 and/or at least one helical groove 82.

FIGS. 2, 3, 4 and 7 to 11 illustrate shroud barrel 80. Shroud barrel 80 as illustrated is located generally around an outside of tubular portion 36 of base 20. Shroud barrel 80 is formed to define at least one blind helical groove 82 that in an embodiment extends at least twenty, e.g., twenty-four, millimeters (about one inch) around an inner surface of the shroud barrel. The end of at least one lug 68 of slider 50 extends into a respective at least one blind helical groove 82 of shroud barrel 80. In the illustrated embodiment, helical grooves 82 are blind and do not extend all the way through the wall of shroud barrel 80, which allows the shroud barrel to better provide a sterile barrier against touch contamination. It should be appreciated however that at least one helical groove 82 does not have to be blind and that the movement of slider 50 during actuation would operate as described if the at least one helical groove instead extended all the way through the wall of shroud barrel 80.

The at least one blind helical groove 82 is oriented in one embodiment such that when the user or patient rotates shroud barrel 80 in a clockwise direction, slider 50 is advanced translationally such that female luer connector 60 of the head 54 of slider 50 is extended towards a connection location with the male luer connector 18, which is the patient line connector. Likewise, when the user or patient rotates the shroud barrel 80 in a counterclockwise direction, slider 50 is reversed translationally such that female luer connector 60 of the head 54 of slider 50 is retracted from the connection location. In an alternative embodiment, the orientation is reversed such that counterclockwise rotation causes slider translation towards connection and clockwise rotation causes slider translation away from connection.

FIG. 8 further illustrates that each blind helical groove 82 may include or define at least one bump or semi-circular protrusion 82b, which extends inwardly into the helical groove. At least one bump or rounded protrusion 82b helps to initiate translational movement of the corresponding lug 68 and provides a smooth ramp/fillet for the lug. At least one bump or semi-circular protrusion 82b located at each end of helical groove 82 also acts as temporary stop or locking feature to releasably maintain the corresponding lug 68 and slider 50 in the fully extended and fully retracted locations discussed below.

In an embodiment, base 20 of transfer set 10 of the present disclosure does not move during activation (or reverse activation). Serrations 24 of larger diameter cylindrical portion 22 of base 20 provide a convenient location for the user or patient to grasp and hold transfer set 10 steady. Slider 50 translates during activation (or reverse activation) relative to base 20. Shroud barrel 80 is rotated by the user or patient's other hand relative to base 20. If tubular portion 36 of base 20 were not provided, the rotation of shroud barrel 80 would simply rotate the at least one engaged lug 68 and the remainder of slider 50 and no translation would occur. The at least one straight through-slot 38 of tubular portion 36 of base 20 however prevents slider 50 from rotating and instead forces slider 50 to translate as the at least one engaged lug 68 moves through the rotating at least one corresponding helical groove 82.

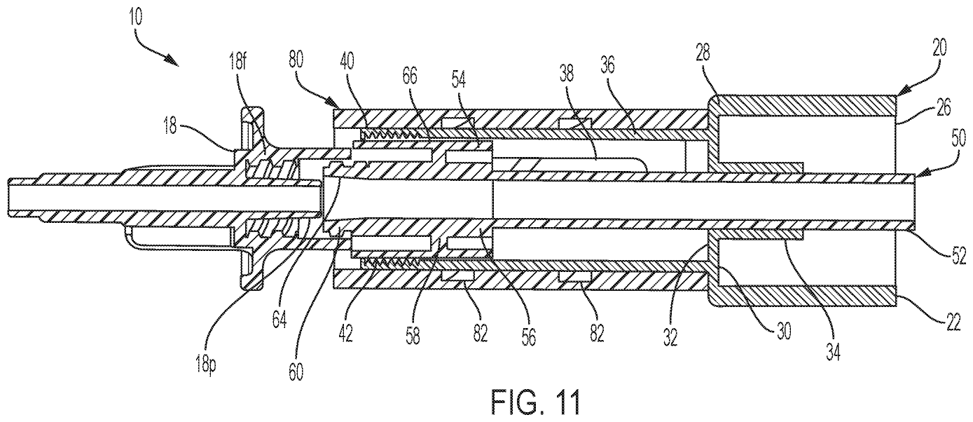

FIG. 2 illustrates a first connection step in which the user or patient removes, e.g., unthreads, minicap 90 (or other cap) from female threads 42 of base 20. In FIG. 2, slider 50 is in a fully retracted position. FIG. 9 illustrates a second connection step in which the user or patient turns or rotates shroud barrel 80 (e.g., in a clockwise direction), moving at least one lug 68 and slider 50 along the at least one helical groove 82, wherein straight slots 38 of base constrain the movement of slider 50 to be a sliding or translational movement. In FIG. 9, slider is translated to the fully extended end of at least one helical groove 82, where it is able to mate with patient connector 18. Here, the user may (i) continue to rotate shroud barrel 80 to thread male luer connector 18 onto female luer connector 60 of slider 50, (ii) rotate male luer connector 18 to thread male luer connector 18 onto female luer connector 60 of slider 50, or (iii) rotate both shroud barrel 80 and male luer connector 18 to thread male luer connector 18 onto female luer connector 60 of slider 50. In any case, in FIG. 10, when male luer connector 18 is fully sealed to female luer connector 60, treatment may proceed in which fresh and used PD fluid travels back and forth through transfer set 10 between PD machine 14 and the patient. In a disconnection step in FIG. 11, after treatment, the user or patient removes (unthreads) male luer connector 18 from female luer connector 60 of slider 50 and rotates shroud barrel 80 in an opposite direction (e.g., counterclockwise) to return slider to the fully retracted position of FIG. 2. The user or patient may then thread a new minicap having an antiseptic agent into threads 42 of base 20.

It should be appreciated that during the above connection and disconnection steps, the mating surfaces of male luer connector 18 and female luer connector 60 are protected from touch contamination by both tubular portion 36 of base 20 and shroud barrel 80. Female luer connector 60 is constantly maintained within tubular portion 36 of base 20 and shroud barrel 80.

It should be understood that various changes and modifications to the presently preferred embodiments described herein will be apparent to those skilled in the art. It is therefore intended that any or all of such changes and modifications may be covered by the appended claims. For example, while transfer set 10 is illustrated showing slider 50 having a female luer connector 60 and the patient connector 18 being a male luer connector, the reverse may be provided alternatively where slider 50 provides a male luer connector, while patient connector 18 is a female luer connector. Also, cap 90 may be any suitable cap and does not have to be a minicap.

The invention is claimed as follows:

1. A peritoneal dialysis (“PD”) transfer set comprising:
a base including at least one slot;
a slider extending within the base, the slider including an elongated tube configured to carry PD fluid, the elongated tube extending to a head of the slider, the head including at least one lug extending through the at least one slot, the head further including a connector for connecting to a mating patient line connector; and
a shroud barrel including at least one helical groove, the shroud barrel extending around a portion of the base so that the at least one helical groove receives the at least one lug, wherein a user may rotate the shroud barrel including the at least one helical groove such that the at least one lug extends along the at least one helical groove, the at least one slot constraining movement of the at least one lug and the slider to being a translational movement.

2. The PD transfer set of claim 1, wherein the base includes a larger diameter cylindrical portion configured for the user to hold while the user rotates the shroud barrel, the base further including a tubular portion defining the at least one slot.

3. The PD transfer set of claim 2, wherein the base includes a port located within the larger diameter cylindrical portion, the port in fluid communication with the elongated tube, the port positioned and arranged to communicate fluidly with patient catheter tubing.

4. The PD transfer set of claim 2, wherein an end of the tubular portion is provided with threads for mating with threads of a cap configured to seal the transfer set prior to treatment.

5. The PD transfer set of claim 2, wherein an inner diameter of the tubular portion is sized to fit closely around the head of the slider.

6. The PD transfer set of claim 2, wherein the tubular portion of the base and the shroud barrel are positioned and arranged to deter touch contamination of a connection between the slider connector and the patient line connector.

7. The PD transfer set of claim 1, wherein the at least one slot is a straight slot.

8. The PD transfer set of claim 1, wherein the connector of the slider is a luer connector.

9. The PD transfer set of claim 1, wherein the slider includes an inner portion providing the connector, a transverse wall extending from the inner portion to a cylindrical wall, the at least one lug extending from the cylindrical wall.

10. The PD transfer set of claim 1, wherein the shroud barrel extends around the tubular portion of the base.

11. The PD transfer set of claim 1, wherein the at least one helical groove is a blind at least one helical groove.

12. The PD transfer set of claim 1, wherein the at least one helical groove is formed on an inner surface of shroud barrel.

13. The PD transfer set of claim 1, wherein the at least one helical groove extends at least at least twenty millimeters along the shroud barrel.

14. The PD transfer set of claim 1, which is configured such that an end of the at least one helical groove is positioned and arranged so as to allow the connector of the slider to meet the patient line connector, and wherein further rotating of the shroud barrel threads the patient line connector into the connector of the slider.

15. The PD transfer set of claim 1, which is configured such that an end of the at least one helical groove is positioned and arranged so as to allow the connector of the slider to meet the patient line connector, and wherein rotating the patient line connector threads the patient line connector into the connector of the slider.

16. The PD transfer set of claim 1, which is configured such that rotating the shroud barrel in a first direction translates the connector of the slider towards the patient line connector and rotating the shroud barrel in a second direction translates the connector of the slider away from the patient line connector.

17. A peritoneal dialysis (“PD”) transfer set comprising:
a base including at least one slot;
a slider extending within the base, the slider configured to carry PD fluid, the slider including at least one lug extending through the at least one slot and a connector for connecting to a mating patient line connector, and
a shroud barrel including at least one helical groove, the shroud barrel extending around a portion of the base so that the at least one helical groove receives the at least one lug, wherein a user may rotate the shroud barrel including the at least one helical groove such that the at least one lug extends along the at least one helical groove, the at least one slot constraining movement of the at least one lug and the slider to being a translating movement, and wherein rotating the shroud barrel in a first direction translates the connector of the slider towards the patient line connector and rotating the shroud barrel in a second direction translates the connector of the slider away from the patient line connector.

18. The PD transfer set of claim 17, wherein the at least one helical groove includes at least one rounded protrusion positioned and arranged to provide a temporary stop that releasably maintains the at least one lug and slider in a location towards the patient line connector or in a location away from the patient line connector.

19. A peritoneal dialysis (“PD”) connection method comprising:
providing a shroud barrel including at least one helical groove;
providing a tubular portion including at least one slot;
providing a connector through which PD fluid flows, the connector including at least one lug extending through the at least one slot and into the least one helical groove; and
enabling the shroud barrel to be rotated to rotate the at least one helical groove so as to cause movement of the at least one lug and the connector, and wherein the at least one slot constrains the movement of the at least one lug and the connector to being a translational movement of the connector towards a mating connector for connection or away from the mating connector after disconnection.

20. The PD connection method of claim 19, which includes enabling the shroud barrel to be rotated in a first direction to translate the connector towards the mating connector for connection and in a second direction to translate the connector away from the mating connector after disconnection.
